(19) United States
(12) Patent Application Publication
(10) Pub. No.: US 2016/0130620 A1
(43) Pub. Date: May 12, 2016

(54) BIOCATALYST FOR SIMULTANEOUSLY DEGRADING LIGNIN AND CELLULOSE, AND METHOD FOR MANUFACTURING HYDROLYSATE AND BIOFUEL USING THE SAME

(71) Applicant: Korea Institute of Science and Technology, Seoul (KR)

(72) Inventors: Yunje KIM, Seoul (KR); Youngsoon UM, Seoul (KR); Han Min WOO, Seoul (KR); Gyeongtaek GONG, Seoul (KR); Kyoungseon MIN, Seoul (KR)

(73) Assignee: Korea Institute of Science and Technology, Seoul (KR)

(21) Appl. No.: 14/934,710

(22) Filed: Nov. 6, 2015

(30) Foreign Application Priority Data
Nov. 7, 2014 (KR) 10-2014-0154354

(51) Int. Cl. C12P 19/14; C12P 19/02

(52) U.S. Cl. CPC C12P 19/14; C12P 19/02

(57) ABSTRACT

The present disclosure relates to a method for simultaneously degrading lignin and cellulose and for boosting effect on the cellulase activity using a specific catalyst. Since the present disclosure allows for the preparation of sugars by degrading not only lignin but also cellulose and hemicellulose using the enzymes which were previously known only as lignin-degrading biocatalysts, it provides the advantage that the preparation of a hydrolysate as a source material for the production of biofuels or biochemicals from lignocellulosic biomass can be simplified and facilitated. As a result, the present disclosure can reduce enzyme cost and can provide improved production efficiency by simplifying the biofuel production process.

### CROSS-REFERENCE TO RELATED APPLICATION

This application claims priority to Korean Patent Application No. 2014-0154354, filed on Nov. 7, 2014, and all the benefits accruing therefrom under 35 U.S.C. §119, the contents of which in its entirety are herein incorporated by reference.

### BACKGROUND

1. Field

The present disclosure discloses a method for simultaneously degrading lignin, cellulose and xylan and for boosting cellulase and xylanase activity by a lignin-degrading enzyme.

Description about National Support Research and Development\

This study is conducted by the support of Korea Ministry of Science, ICT and Future Planning under the supervision of Korea Institute of Science and Technology, and research title is development of lignin degradation technique for securing ligno-biofuel source (Research management agency: National Research Foundation of Korea, Grant Number: 1711002201).

2. Description of the Related Art

At present, the humankind faces the problems of depletion of petroleum resources and global warming. With the increasing global interest in new renewable energy for replacing fossil fuels and solving the global warming problem, the biorefinery for producing fuels and high-value-added compounds using environment-friendly biological resources instead of petroleum is welcomed as a new paradigm. In this regard, methods for producing biofuels and biochemicals from non-edible lignocellulosic biomass, instead of food resources such as corn, are actively being developed. Developed countries including the US are making efforts to secure energy security in a nationwide level by increasing biofuel production from lignocellulosic biomass in the long term.

The production of biofuels and biochemicals in the biorefinery using lignocellulosic biomass as substrate with microorganisms generally follows the process of pretreatment for degrading lignin, saccharification for obtaining fermentable sugars (degradation of cellulose and hemicellulose), fermentation by microorganisms, and separation and purification of metabolites. The pretreatment processes for degrading or loosening lignin includes steam explosion, dilute acid or alkali treatment, microwave irradiation, ionizing radiation, hydrolythermolysis, etc. and biological pretreatment techniques use lignin-degrading microorganisms secreting various biocatalysts. The microorganisms are known to secrete biocatalysts such as lignin peroxidase, manganese peroxidase, copper oxidase, etc.

In the saccharification processes for obtaining sugars (glucose, xylose, etc.) from biomass for microbial fermentation, various cellulases and xylanases are used in combination in general. In order to degrade cellulose to monosaccharides, the activity of endo-glucanase, exo-glucanase and β-exo-glucanase is necessary at the same time. However, since the cellulose-degrading enzymes have poor stability and show decreased activity due to product inhibition, the biocatalysts have to be loaded in large quantities. This leads to increased cost, which makes industrial application difficult. Therefore, development of a multifunctional cellulose-degrading enzyme which has superior stability is necessary.

Recently, proteins that enhance the cellulase activity in cellulose degradation were discovered. Chitin-binding protein, glycoside hydrolase family 61 (GH61), expansin, etc. are known to enhance or boost cellulase activity when mixed with cellulase, thereby leading to production of more fermentable sugars necessary for microbial fermentation. However, the reaction mechanism is not fully understood yet. And, these proteins cannot degrade cellulose on their own but merely enhance the degradation of cellulose by assisting cellulase.

### SUMMARY

The present disclosure is directed to providing a method for simultaneously degrading lignin and cellulose and for boosting the activity of a cellulase using a lignin-degrading biocatalyst.

In an exemplary embodiment, the present disclosure provides a method for obtaining sugars derived from lignocellulosic biomass, the method including: treating lignocellulosic biomass containing cellulose and hemicellulose with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase, wherein the sugar includes one or more sugar selected from a group consisting of fermentable sugars from cellulose and fermentable sugars from hemicellulose.

In another exemplary embodiment, the present disclosure provides a method for boosting the cellulose or xylanase activity, the method including: treating a cellulase or xylanase with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase, which is known to be involved in the degradation of lignin and is also demonstrated to boost the cellulase or xylanase activity through the present disclosure.

In another exemplary embodiment, the present disclosure provides a method for degrading lignin, cellulose and hemicellulose at the same time, including treating lignocellulosic biomass containing lignocellulose with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase, and a method for producing bioenergy using the resulting sugars.

According to an exemplary embodiment of the present disclosure, sugars can be prepared by degrading not only lignin but also cellulose and hemicellulose using an oxidoreductase previously known to degrade lignin only. While the one or more catalyst selected from a group consisting of LiP, MnP, DyP, VP, SOD and laccase is an oxidase, it can produce fermentable sugars. Therefore, according to an exemplary embodiment of the present disclosure, a process for preparing a sugar as a source material for the production of a biofuel or a biochemical from lignocellulosic biomass can be simplified. That is to say, a hydrolysate can be prepared by degrading lignin, cellulose and hemicellulose at the same time. Therefore, according to an exemplary embodiment of the present disclosure, use of the existing polysaccharide-decomposing hydrolase such as cellulase or xylanase can be decreased and biofuel production efficiency can be improved by simplifying the production process. Also, according to an exemplary embodiment of the present disclosure, since the activity of cellulase and xylanase can be enhanced using the enzyme, a sugar as a source material for the production of a biofuel or a biochemical from lignocellulosic biomass can be produced more effectively.

### BRIEF DESCRIPTION OF THE DRAWINGS

FIG. 1 shows the production of sugars from carboxymethyl cellulose (CMC) as a substrate by lignin peroxidase (LiP) and manganese peroxidase (MnP) according to an exemplary embodiment of the present disclosure.

FIG. 2 shows the production of sugars from xylan as a substrate by lignin peroxidase (LiP) and manganese peroxidase (MnP) according to an exemplary embodiment of the present disclosure.

FIG. 3 shows the relative activity of lignin peroxidase (LiP) depending on pH when carboxymethyl cellulose (CMC), p-nitrophenyl cellobiose (pNPC), cellobiose and xylan were used as substrates according to an exemplary embodiment of the present disclosure.

FIG. 4 shows the relative activity of lignin peroxidase (LiP) depending on temperature when carboxymethyl cellulose (CMC), p-nitrophenyl cellobiose (pNPC), cellobiose and xylan were used as substrates according to an exemplary embodiment of the present disclosure.

FIG. 5 shows the relative activity of manganese peroxidase (MnP) depending on pH when carboxymethyl cellulose (CMC), p-nitrophenyl cellobiose (pNPC), cellobiose and xylan were used as substrates according to an exemplary embodiment of the present disclosure.

FIG. 6 shows the relative activity of manganese peroxidase (MnP) depending on temperature when carboxymethyl cellulose (CMC), p-nitrophenyl cellobiose (pNPC), cellobiose and xylan were used as substrates according to an exemplary embodiment of the present disclosure.

FIG. 7 shows the degradation activity of lignin peroxidase (LiP) and manganese peroxidase (MnP) when carboxymethyl cellulose (CMC), p-nitrophenyl cellobiose (pNPC), cellobiose, regenerated amorphous cellulose (RAC), Avicel and xylan were used as substrates according to an exemplary embodiment of the present disclosure.

FIG. 8 shows that cellobiose is degraded by manganese peroxidase (MnP) and glucose is produced quantitatively as a fermentable sugar when cellobiose was used as a substrate according to an exemplary embodiment of the present disclosure.

### DETAILED DESCRIPTION

In an aspect, the present disclosure provides a method for obtaining sugars derived from lignocellulosic biomass, including treating lignocellulosic biomass containing lignin, cellulose and lignocellulose with one or more catalyst selected from a group consisting of lignin peroxidase (LiP; E.C. 1.11.1.14), manganese peroxidase (MnP; E.C. 1.11.1.13), heme-containing dye-decolorizing peroxidase (DyP; E.C. 1.11.1.19), versatile peroxidase (VP; E.C. 1.11.1.16), superoxide dismutase (SOD: E.C. 1.15.1.1) and laccase (E.C. 1.10.3.2). The sugar may include one or more sugar selected from a group consisting of fermentable sugars from cellulose and fermentable sugars from hemicellulose.

As used herein, the term “biomass” refers to a biological material derived from plants that can be used as a chemical energy source and means lignocellulosic biomass composed of lignin, cellulose and hemicellulose, such as grass, wood and agricultural waste such as rice straw.

As used herein, the term “lignocellulosic biomass” refers to biomass derived from plant, specifically woody plant, or the plant having hard and enlarged stem and root as opposed to grass. Since the lignocellulosic biomass contains cellulose and lignocellulose in large quantity, it may be used as a sugar as a source material for bioenergy production.

As used herein, the term “fermentable sugar” can refer to sugars obtained from degradation of the polymer cellulose or hemicellulose and utilized for microbial fermentation. The cellulose is a linear-chain polysaccharide consisting of glucose units linked by β-1,4 linkages. Because it has a much stronger physical and chemical structure than amylose wherein glucose units are bound through α-1,4 linkages, it is relatively difficult to be degraded. The hemicellulose is a polymer consisting mainly of the five-carbon sugar xylose and arabinose, mannose, galactose or glucose. It has a low degree of polymerization as compared to the cellulose. Because of the low degree of polymerization and structural regularity as compared to the cellulose, it is relatively easy to degrade. Specifically, the fermentable sugar from cellulose may be glucose and the fermentable sugars from hemicellulose may be one or more of xylose, arabinose, mannose, and galactose, although not being limited thereto.

As used herein, the term “hydrolysate” refers to a solution containing sugars obtained by degrading cellulose or hemicellulose, which is a sugar-based polymer.

The lignin peroxidase (LiP) and the manganese peroxidase (MnP) may be derived from fungi such as Phanerochaete chrysosporium, although not being limited thereto. The laccase is a copper-containing polyphenol oxidase and may be derived from fungi such as Pleurotus ostreatus although not being limited thereto. When an oxygen molecule is reduced to a water molecule, it may form radicals by releasing electrons from polyphenols, methoxylated monophenols, aromatic amines, etc.

As used herein, the term “lignocellulose” refers to a structural component of a plant material that is composed of cellulose, hemicellulose and lignin. The lignin is a polymer of methoxylated p-coumaryl alcohol, coniferyl alcohol, sinapyl alcohol, etc. It is a hydrophobic and complex polymer containing various aromatic compounds. The lignin is chemically very resistant and is difficult to degrade. In lignocellulosic biomass, lignin is covalently bonded to hemicellulose and hemicellulose is bonded to cellulose via hydrogen bonding. Overall, the lignocellulosic biomass has a structure in which linear cellulose microfibrils are surrounded by hemicellulose via hydrogen bonding and, in turn, the hemicellulose is surrounded by lignin via covalent bonding.

Therefore, to obtain sugars from the lignocellulosic biomass, the lignin surrounding the lignocellulose has to be degraded first and, because the chemical process at high temperature and high pressure for degrading lignin is different from the enzymatic process for degrading cellulose (and hemicellulose), the degradation has to be carried out through different steps.

In contrast, the method for obtaining sugars derived from lignocellulosic biomass according to the present disclosure is convenient and economical since the degradation of lignin and cellulose and hemicellulose can be carried out using the above-described catalysts.

As used herein, the cellulose (or hemicellulose) may be, for example, carboxymethyl cellulose (CMC), Avicel, cellobiose, p-nitrophenyl cellobioside, regenerated amorphous cellulose (RAC), xylan (from beech wood), etc., although not being limited thereto.

In the method for obtaining sugars derived from lignocellulosic biomass according to an exemplary embodiment of the present disclosure, when the catalyst is one or more of lignin peroxidase (LiP), manganese peroxidase (MnP), heme-containing dye-decolorizing peroxidase (DyP) and versatile peroxidase (VP), the catalyst may be treated together with hydrogen peroxide. The order of treatment with the catalyst and the hydrogen peroxide is not particularly limited. Also, they may be treated simultaneously. Also, the catalyst may be treated together with Mn²⁺ and hydrogen peroxide. The order of treatment with the catalyst, the Mn²⁺ and the hydrogen peroxide is not particularly limited. Also, they may be treated simultaneously.

The addition of hydrogen peroxide is a novel method which has not been introduced in degradation using cellulase and chitin-binding protein, glycoside hydrolase family 61 (GH61), expansin, etc. known to enhance or boost cellulase activity. In an exemplary embodiment, Mn²⁺ may be added when the catalyst is MnP. The Mn²⁺ may be added to other proteins, too.

In the method for obtaining sugars derived from lignocellulosic biomass according to an exemplary embodiment of the present disclosure, the fermentable sugar from cellulose may be glucose and the fermentable sugars from hemicellulose may be one or more of xylose, arabinose, mannose, and galactose.

In the method for obtaining sugars derived from lignocellulosic biomass according to an exemplary embodiment of the present disclosure, the sugar derived from lignocellulosic biomass may be included in a hydrolysate derived from lignocellulosic biomass, the hydrolysate may further contain a degradation product of lignin and the degradation product may contain one or more compound selected from a group consisting of methoxylated coumaryl alcohol, coniferyl alcohol and sinapyl alcohol. It is because the catalyst used in the present disclosure is for degrading lignin.

The method for obtaining sugars derived from lignocellulosic biomass according to an exemplary embodiment of the present disclosure may further comprise a step of treating cellulase or xylanase with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase. When the polysaccharide-decomposing hydrolase cellulase or xylanase is treated with the one or more catalyst, the activity of the cellulase or xylanase may be enhanced.

The inventors of the present disclosure have found out that the above-described catalysts can not only degrade cellulose (and hemicellulose, i.e., xylanase) but also boost the activity of a cellulase (and hemicellulase). Therefore, when sugars are obtained from lignocellulosic biomass using the catalyst, it is very useful, because the treatment with the catalyst leads to simultaneous degradation of lignin and cellulose (and hemicellulose) as well as the enhancement of the activity of the cellulase (and hemicellulase), the saccharification efficiency can be improved while reducing the use of the expensive cellulase (and hemicellulase).

In an exemplary embodiment of the present disclosure, the cellulase may be one or more enzyme selected from a group consisting of endo-glucanase, exo-glucanase, cellobiohydrolase, cellobiose dehydrogenase and β-glucosidase. However, without being limited thereto, any enzyme that can degrade cellulose (and hemicellulose) may be used.

In another aspect, the present disclosure relates to a method for simultaneously degrading lignin, cellulose and hemicellulose, including treating lignocellulosic biomass containing lignocellulose with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase.

In another aspect, the present disclosure relates to a method for producing bioenergy, including: obtaining sugars including fermentable sugars from cellulose and fermentable sugars from hemicellulose by treating lignocellulosic biomass containing lignocellulose with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase; and producing bioenergy using the sugar. The second step, i.e., the production of bioenergy from the sugar may be carried out by any method known in the art. Specifically, the sugar may be used as the carbon source for microbial fermentation, although not being limited thereto.

In the various aspects of the present disclosure, the concentration of the catalyst may be suitably adjusted by those skilled in the art. Also, the treatment temperature and pH may be adjusted depending on the catalyst. For example, when LiP, MnP and DyP are used as the catalyst, the temperature may be 20-60° C. and the pH may be 2-5. More specifically, the temperature may be 45-60° C., 50-60° C. or 50° C. And, if the catalyst is laccase, the temperature may be 20-80° C. and the pH may be 2-10, although not being limited thereto.

Hereinafter, the present disclosure will be described in detail through examples. However, the following examples are for illustrative purposes only and it will be apparent to those of ordinary skill in the art that the scope of the present disclosure is not limited by the examples.

### EXAMPLE 1

### Degradation of Cellulose and Hemicellulose

In order to confirm whether the biocatalyst for simultaneously degrading lignin and cellulose according to the embodiments of the present disclosure can degrade cellulose and hemicellulose on its own, sugars produced from degradation of cellulose and hemicellulose was measured.

As the biocatalyst for simultaneously degrading lignin and cellulose, lignin peroxidase (LiP) and manganese peroxidase (MnP) derived from Phanerochaete chrysosporium (Sigma) were used. As a cellulose substrate, 5 g/L carboxymethyl cellulose (Sigma) was used and, as a hemicellulose substrate, 2.5 g/L xylan (from beech wood) (Sigma) was used. For the LiP reaction, 2.5 mg/mL LiP and 0.1 mM hydrogen peroxide were added to the substrate. And, for the MnP reaction, 2.5 mg/mL MnP, 0.1 mM hydrogen peroxide and 2 mM MnSO₄ were added to the substrate. The mixture was incubated at 30° C. at pH 4.5 for 24 hours. The production of fermentable sugars was measured by the DNS method. The DNS solution consisted of 10 g/L NaOH, 5 g/L DNS (3,5-dinitrosalicylic acid), 1 g/L phenol and 100 g/L Rochelle salt. For analysis, 250 μL of the analyte and 750 μL of the DNS solution were mixed and boiled for 5 minutes. After sufficiently cooling at room temperature, absorbance was measured at 540 nm. For the cellulose and hemicellulose substrates, the concentration of fermentable sugars was normalized to that of standard glucose and xylose solutions, respectively.

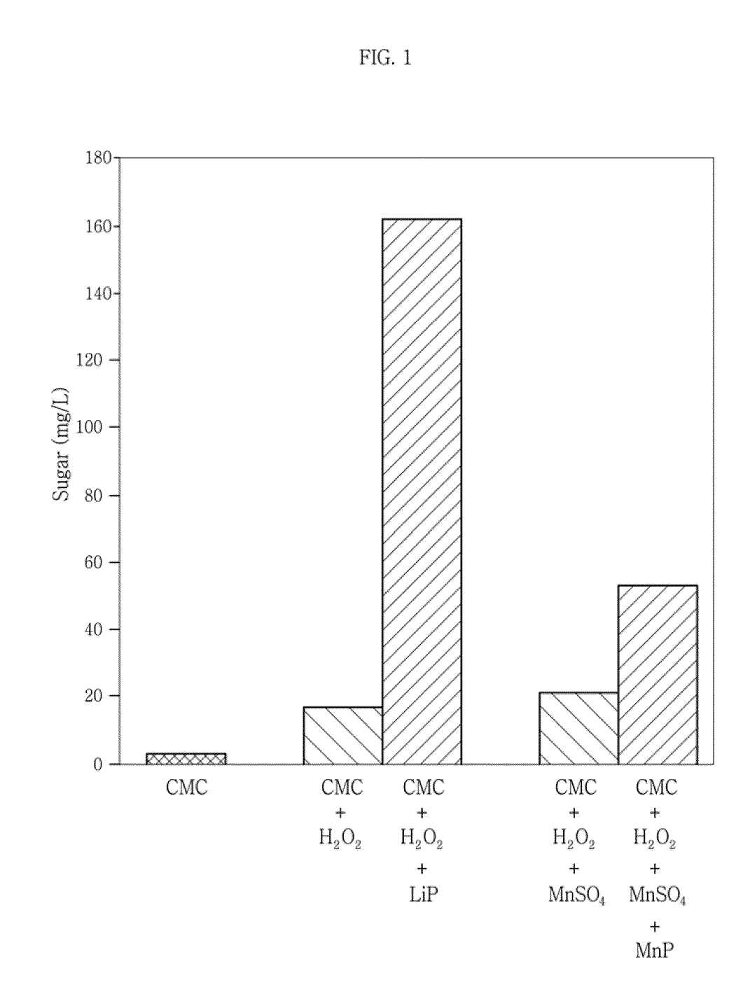

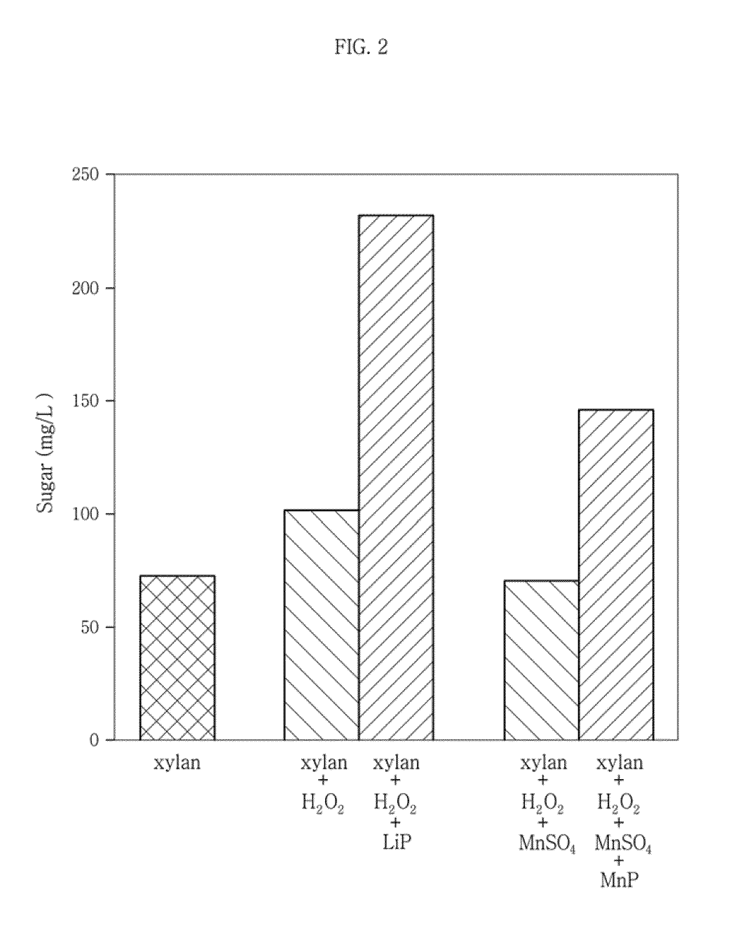

The result of degrading carboxymethyl cellulose as the cellulose is shown in FIG. 1. It can be seen that LiP and MnP can produce sugars by degrading carboxymethyl cellulose, as compared to the biocatalyst-free control group. The result of degrading xylan as the hemicellulose is shown in FIG. 2. It can be seen that LiP and MnP can produce sugars by degrading xylan, as compared to the biocatalyst-free control group.

### EXAMPLE 2

### Analysis of Optimal Temperature and pH

Since it was confirmed that LiP and MnP degrade carboxymethyl cellulose and xylan, experiment was conducted to investigate the optimal temperature and pH. In addition, it was investigated whether LiP and MnP also degrade cellobiose and p-nitrophenyl cellobiose and what the optimal temperature and pH are for them.

Experiment was conducted for 24 hours while varying pH from 2.5 to 6.0 using an acetate buffer and a phosphate buffer. Temperature was varied from 30° C. to 70° C.

As the biocatalyst for simultaneously degrading lignin and cellulose, lignin peroxidase (LiP) and manganese peroxidase (MnP) derived from Phanerochaete chrysosporium (Sigma) were used. As substrates, carboxymethyl cellulose, cellobiose, p-nitrophenyl cellobiose and xylan, 1 g/L each, were used. For the LiP reaction, 2.5 mg/mL LiP and 0.1 mM hydrogen peroxide were added. And, for the MnP reaction, 2.5 mg/mL MnP, 0.1 mM hydrogen peroxide and 2 mM MnSO₄ were added. The production of fermentable sugars from xylan or carboxymethyl cellulose was measured by the DNS method. The production of glucose from cellobiose was analyzed by liquid chromatography (Agilent model 1200). A refractive index detector and an Aminex HPX-87H column were used. The production of p-nitrophenol from p-nitrophenyl cellobiose was analyzed by measuring absorbance at 410 nm using a spectrophotometer (Cary60, Agilent Technology).

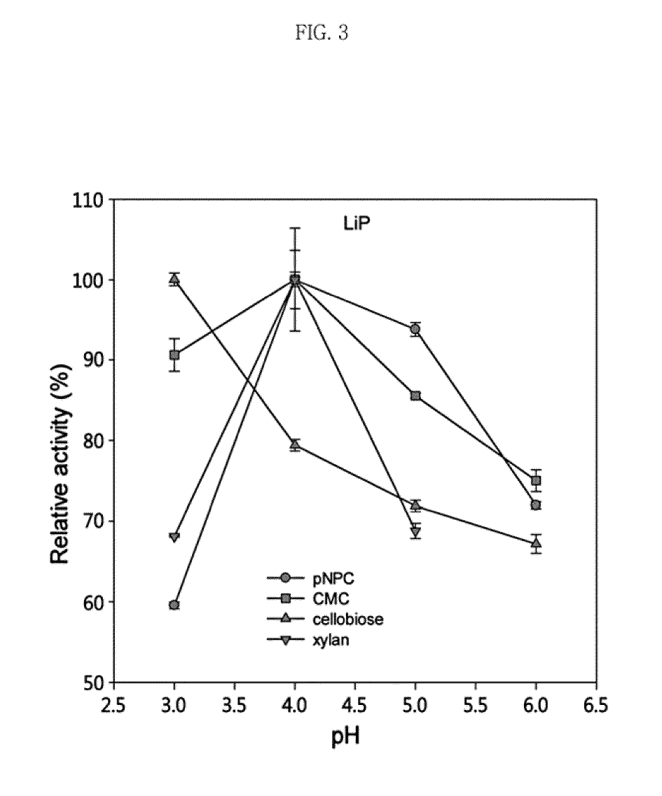

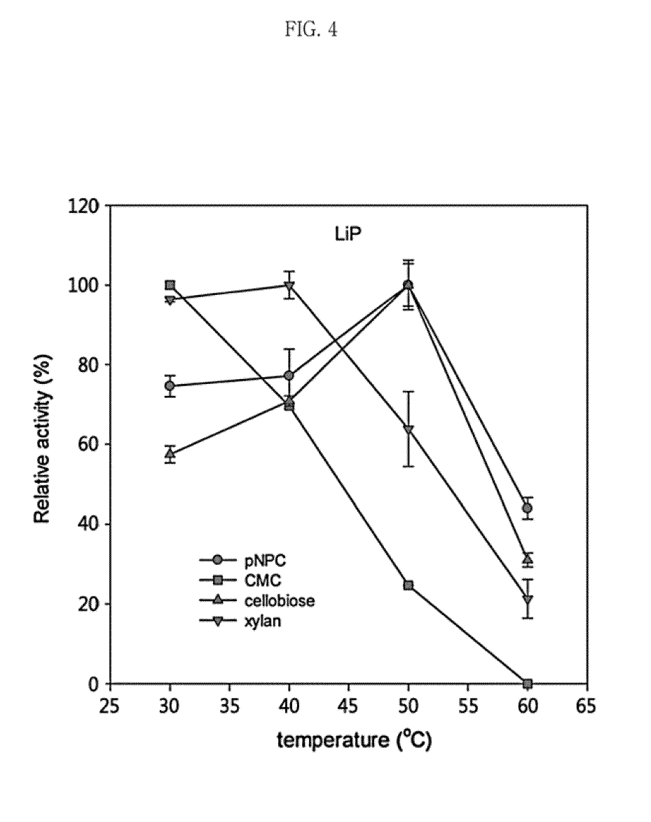

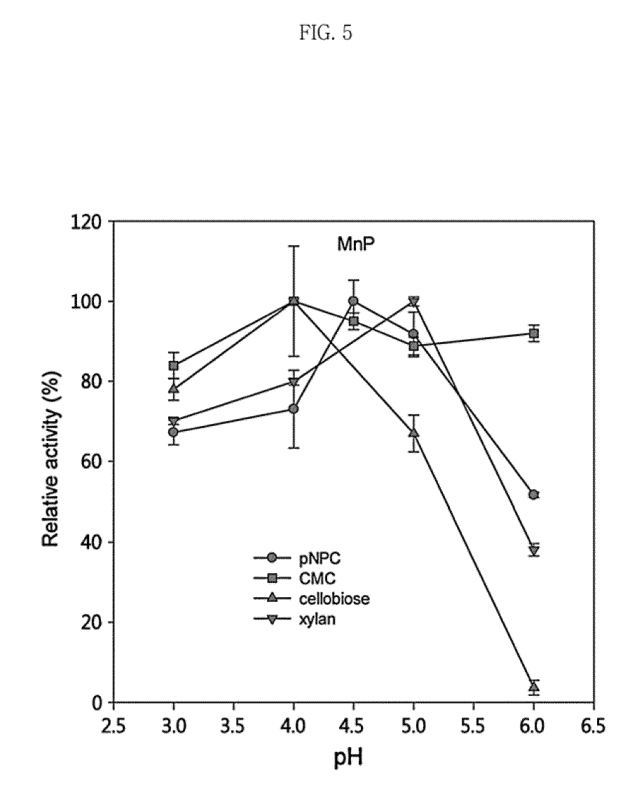

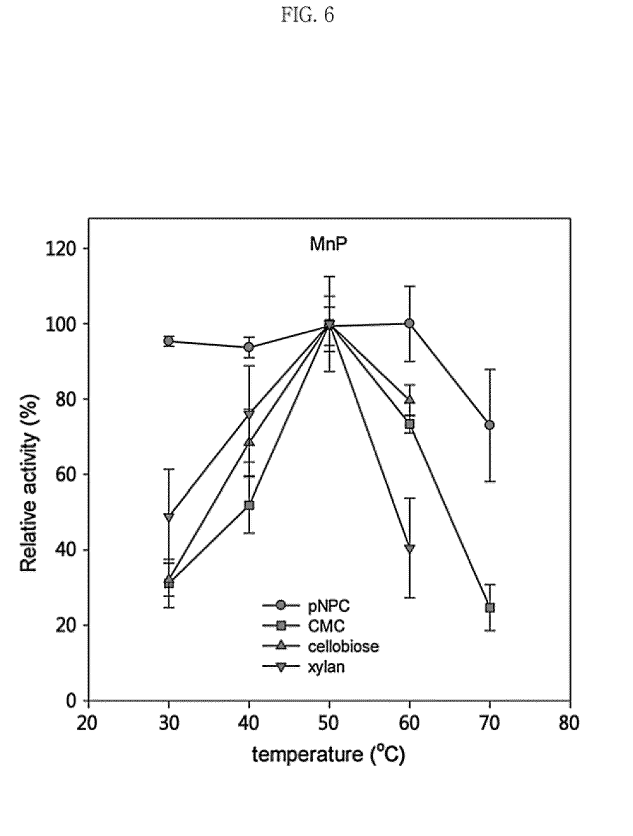

After analyzing the concentration of the reaction product from each substrate, relative degradation activity was calculated as a function of temperature and pH. The result is shown in FIGS. 3-6.

As can be seen from FIGS. 3-6, the optimal temperature for degradation of p-nitrophenyl cellobiose by LiP and degradation of carboxymethyl cellulose, cellobiose, p-nitrophenyl cellobiose and xylan by MnP was 50° C. Considering that the optimal pH and temperature of LiP and MnP for the reaction with the reference substrate veratryl alcohol and Mn²⁺0 ion are 4-4.5 and 30° C., respectively, the change in the optimal temperature and activity depending on the substrate is a very peculiar characteristic.

### EXAMPLE 3

### Cellulose and Hemicellulose Degradation Activity

The cellulose and hemicellulose degradation activity of the biocatalyst for simultaneously degrading lignin and cellulose according to the embodiments of the present disclosure was measured.

As the biocatalyst for simultaneously degrading lignin and cellulose, lignin peroxidase (LiP) and manganese peroxidase (MnP) derived from Phanerochaete chrysosporium (Sigma) were used. As cellulose substrates, carboxymethyl cellulose, Avicel, cellobiose, nitrophenyl cellobiose and 1 g/L regenerated amorphous cellulose, were used. As a hemicellulose substrate, 1 g/L xylan was used. For the LiP reaction, 2.5 mg/mL LiP and 0.1 mM hydrogen peroxide were added to the substrate. And, for the MnP reaction, 2.5 mg/mL MnP, 0.1 mM hydrogen peroxide and 2 mM MnSO₄ were added to the substrate. The production of sugars from the carboxymethyl cellulose, Avicel, regenerated amorphous cellulose and xylan was measured by the DNS method. The production of glucose from the cellobiose was analyzed by liquid chromatography (Agilent model 1200). A refractive index detector and an Aminex HPX-87H column were used. The production of p-nitrophenol from the p-nitrophenyl cellobiose was analyzed by measuring absorbance at 410 nm using a spectrophotometer (Cary60, Agilent Technology). The reaction was conducted for 24 hours at the optimal pH and temperature shown in FIGS. 3-6.

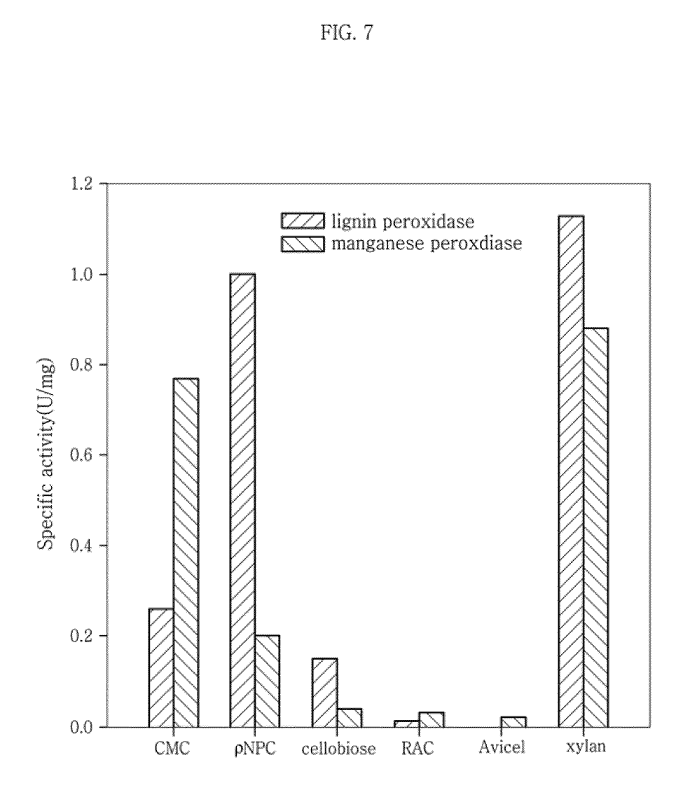

The concentration of the reaction product from each substrate was analyzed and degradation activity was calculated therefrom. The result is shown in FIG. 7. It was confirmed that LiP and MnP had endo-glucanase, exo-glucanase, β-glucosidase and xylanase activities, which are necessary to degrade (hemi)cellulose to monosaccharides. In FIG. 7, the activity unit (U) is defined as the amount of the biocatalyst required to produce 1 μmole of product in 1 minute. It can be seen from FIG. 7 that LiP and MnP can degrade cellulose and hemicellulose on their own.

### EXAMPLE 4

### Enhancement of Activity of Cellulase and Xylanase

The boosting effect of the activity of cellulase and xylanase by the biocatalyst for simultaneously degrading lignin and cellulose according to the embodiments of the present disclosure was evaluated.

As the biocatalyst for simultaneously degrading lignin and cellulose, lignin peroxidase (LiP) and manganese peroxidase (MnP) derived from Phanerochaete chrysosporium (Sigma) were used. As the cellulase, 1 unit of a cellulase derived from Trichoderma reesei (ATCC26921, Sigma) was used and, as the xylanase, 0.25 unit of a xylanase derived from Thermomyces lanuginosus (Sigma) was used. As substrates for testing the activity enhancement, carboxymethyl cellulose (CMC) and Avicel (1 g/L and 10 g/L) and xylan (2.5 g/L) were used. The produced fermentable sugar was analyzed by the DNS method as in Example 1. For the LiP reaction, 2.5 mg/mL LiP and 0.1 mM hydrogen peroxide were added to the substrate. And, for the MnP reaction, 2.5 mg/mL MnP, 0.1 mM hydrogen peroxide and 2 mM MnSO₄ were added. The reaction was conducted for 24 hours at the optimal pH and temperature shown in FIGS. 3-6.

The effect of enhancing the activity of cellulase and xylanase by LiP and MnP was calculated according to Equation 1. The result is shown in Table 1.

DS(degree of synergism)=(fermentable sugars production when cellulase and peroxidase were used together)/(fermentable sugars production when only cellulase was used+fermentable sugars production when only peroxidase was used) [Equation 1]

TABLE 1

| | | Substrate | Fermentable sugar (gL⁻¹) | | | |
|---|---|---|---|---|---|---|
| Biocat- | Sub- | conc. | | Perox- | Cellulase + | |
| alyst | strate | (gL⁻¹) | Cellulase | idase | peroxidase | DS* |
| LiP | CMC | 1.0 | 0.633 | 0.213 | 0.915 | 1.08 |
| | | 10.0 | 1.214 | 0.229 | 1.828 | 1.27 |
| MnP | CMC | 1.0 | 0.538 | 0.253 | 0.920 | 1.16 |
| | | 10.0 | 1.147 | 0.195 | 2.002 | 1.49 |
| | Avicel | 1.0 | 0.530 | 0.198 | 1.000 | 1.37 |
| | | 10.0 | 0.713 | 0.213 | 1.735 | 1.87 |
| | Xylan | 2.5 | 0.553 | 0.134 | 0.702 | 1.02 |

For carboxymethyl cellulose (CMC), the sugar production was increased by 27% when LiP and cellulase were used together as compared to when only cellulase was used. And, as for the cellulase activity enhancement by MnP, the sugar production from carboxymethyl cellulose (CMC) and Avicel as substrates was increased by 49% and 87%, respectively, when MnP and cellulase were used together as compared to when only cellulase was used. And, when xylan was treated with xylanase and MnP together, the fermentable sugar production was increased by 2%. Accordingly, it can be seen from Table 1 that, for the substrates carboxymethyl cellulose (CMC) and Avicel, lignin peroxidase (LiP) and manganese peroxidase (MnP) increase fermentable sugar production by boosting the activity of cellulase.

### EXAMPLE 5

### Cellobiose Degradation Activity

The degradation product of cellobiose by the biocatalyst for simultaneously degrading lignin and cellulose according to the embodiments of the present disclosure was measured.

As the biocatalyst for simultaneously degrading lignin and cellulose, manganese peroxidase (MnP) derived from Phanerochaete chrysosporium (Sigma) was used. For the reaction, 0.1 mM hydrogen peroxide and 2 mM MnSO₄ were added to 1 g/L cellobiose.

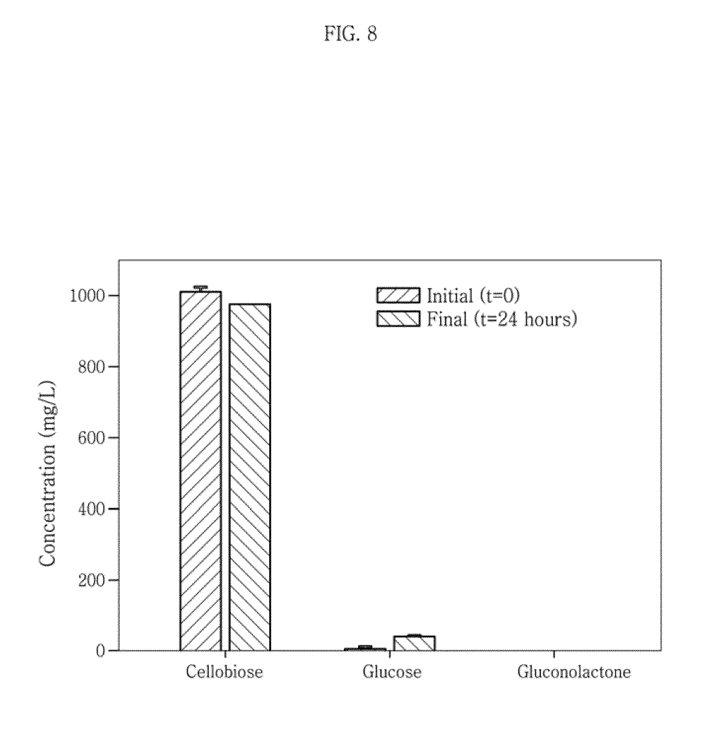

The fermentable sugars produced by MnP were analyzed by the DNS method because the DNS method is most widely used to quantify fermentable sugars obtained from saccharification. However, the DNS method is not suitable for analyzing oxidized sugars (e.g., gluconolactone) obtained from oxidative degradation. To identify whether any form of sugar was produced by the MnP-driven cellobiose degradation, catalytic products of cellobiose were analyzed by high-performance liquid chromatography (Agilent model 1200). A refractive index detector and an Aminex HPX-87H column were used. The result is shown in FIG. 8.

It was confirmed that cellobiose (102 μM) was degraded and converted quantitatively to glucose (205 μM). Interestingly, although MnP is an oxidase, not a hydrolase, cellobiose was converted to the fermentable sugar glucose and gluconolactone, which is an oxidized form of glucose, was not detected. This reveals that MnP degrades cellobiose into a fermentable sugar as if it were a hydrolase.

While the exemplary embodiments have been shown and described, it will be obvious to those skilled in the art that they are only exemplary and do not limit the scope of the present disclosure. Therefore, the essential scope of the present disclosure is defined by the appended claims and equivalents thereof.

What is claimed is:

1. A method for obtaining sugars derived from lignocellulosic biomass, the method comprising:
treating lignocellulosic biomass comprising cellulose and hemicellulose with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase,
wherein the sugar comprises one or more sugar selected from a group consisting of fermentable sugars from cellulose and fermentable sugars from hemicellulose.

2. The method according to claim 1, wherein, when the catalyst comprises one or more of lignin peroxidase (LiP), manganese peroxidase (MnP), heme-containing dye-decolorizing peroxidase (DyP) and versatile peroxidase (VP), the catalyst is treated together with hydrogen peroxide.

3. The method according to claim 1, which further comprises treating with Mn²⁺.

4. The method according to claim 1, wherein the fermentable sugar from cellulose is glucose and the fermentable sugar from hemicellulose is one or more of xylose, arabinose, mannose, and galactose.

5. The method according to claim 1, wherein the sugar derived from lignocellulosic biomass is included in a hydrolysate derived from lignocellulosic biomass, the hydrolysate further comprises a degradation product of lignin and the degradation product comprises one or more compound selected from a group consisting of methoxylated coumaryl alcohol, coniferyl alcohol and sinapyl alcohol.

6. The method according to claim 1, wherein the method is a method for simultaneously degrading lignin, cellulose and hemicelluloses, which are comprised in the lignocellulosic biomass.

7. The method according to claim 1, which further comprises treating a cellulase or xylanase with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase.

8. The method according to claim 7, wherein the cellulase comprises one or more enzyme selected from a group consisting of endo-glucanase, exo-glucanase, cellobiohydrolase, cellobiose dehydrogenase and β-glucosidase.

9. The method according to claim 7, wherein the catalyst boosts the activity of a cellulase or xylanase.

10. A method for boosting cellulase or xylanase activity, the method comprising treating a cellulase or xylanase with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase.

11. The method according to claim 10, wherein the cellulase comprises one or more enzyme selected from a group consisting of endo-glucanase, exo-glucanase, cellobiohydrolase, cellobiose dehydrogenase and β-glucosidase.

12. A method for producing bioenergy, the method comprising:
obtaining a sugar comprising fermentable sugars from cellulose and fermentable sugars from hemicellulose by treating lignocellulosic biomass comprising lignocellulose with one or more catalyst selected from a group consisting of lignin peroxidase (LiP), manganese peroxidase (MnP), heme-containing dye-decolorizing peroxidase (DyP), versatile peroxidase (VP), superoxide dismutase (SOD) and laccase; and
producing bioenergy using the sugar.
